(19) United States
(12) Patent Application Publication
(10) Pub. No.: US 2024/0303544 A1
(43) Pub. Date: Sep. 12, 2024

(54) GRAPH DATABASE TECHNIQUES FOR MACHINE LEARNING

(71) Applicant: The Regents of the University of California, Oakland, CA (US)

(72) Inventors: Sergio E. Baranzini, Oakland, CA (US); Charlotte A. Nelson, Oakland, CA (US)

(21) Appl. No.: 18/551,483

(22) PCT Filed: Mar. 29, 2022
(86) PCT No.: PCT/US2022/022298

§ 371 Date: Sep. 20, 2023

Related U.S. Application Data
(60) Provisional application No. 63/167,479, filed on Mar. 29, 2021.

(51) Int. Cl. G06N 20/00

(52) U.S. Cl. CPC G06N 20/00

(57) ABSTRACT

A process is provided for using a graph database (e.g., SPOKE) to generate training vectors (SPOKEsigs) and train a machine learning model to classify biological entities. A cohort's input data records (EHRs) are compared to graph database nodes to identify overlapping concepts. Entry nodes (SEPs) associated with these overlapping concepts are used to generate propagated entry vectors (PSEVs) that encode the importance of each database node for a particular cohort, which helps train the model with only relevant information. Further, the propagated entry vectors for a given entity with a known classification can be aggregated to create training vectors. The training vectors are used as inputs to train a machine learning model. Biological entities with an unknown classification can be classified with a trained machine learning model. Entity signature vectors are generated for entities without a classification and input into the trained machine learning model to obtain a classification.

### BACKGROUND

Classification and prediction of biomedical outcomes (e.g., patient response) and of drug targets and their mechanism of action (e.g., molecular pathways) is a difficult problem. It is desirable to use as much data as possible when training a machine learning model (e.g., a classification model), but it is difficult to identify which data to use and how to assemble into a computer-readable input feature vector. For instance, understanding a drug's effectiveness for a particular patient may involve information about symptoms, genotype, and nutrition. As another example, a proper diagnosis of a patient may ideally account for many types of data.

Further, typical machine learning techniques do not account for interactions between the data in the input feature vector, but instead rely on the machine learning model to account for any such interactions. Using such large amounts of data and relying exclusively on a machine learning model can lead to inaccuracies and instabilities in training and testing a given model.

Accordingly, new machine learning techniques that use large amounts of input data in a biologically meaningful manner are needed.

### BRIEF SUMMARY

Techniques are described herein for training a model for classifying biological entities and for classifying biological entities using a machine learning model. The techniques include using a graph database (e.g., SPOKE) to generate training vectors (also referred to herein as SPOKEsigs) for each biological entity with a known classification. Generating training vectors involves identifying entity record fields (EHR fields) from the entity's records (EHRs) and linking these to corresponding graph database concepts (nodes). For each overlapping concept (SEP), an entry vector (PSEV), encoding the importance of each graph database node, is calculated using a modified random walk algorithm. For an individual entity, each entry vector associated with the SEPs found in an entity's records is summed to produce a training vector. A machine learning model is trained by inputting the training vectors into a machine learning model and modifying model variables until the model outputs the training vector's known classification. Once the model is trained, entity signature vectors are generated like training vectors for biological entities with an unknown classification. The entity signature vectors are then input into the trained machine learning model and a classification (e.g. a disease diagnosis) is output from the model.

In an illustrative embodiment, a machine learning model for classifying biological entities can be trained. As part of the training, a graph database is stored comprising (1) M nodes of a plurality of node types and (2) a plurality of edges of a plurality of edge types. A plurality of entity records are received for a plurality of entities, each with a plurality of fields and a known classification. N entry nodes of the M nodes that each match one of the plurality of fields are identified, wherein N is less than M. For each of the N entry nodes, a propagated entry vector having M entry values is generated, wherein each of the M entry values represents an importance of a corresponding node to the entry node. For each of the plurality of entities: each of the fields of the corresponding entity record that matches one of the N entry nodes is identified, thereby identifying K entity-specific entry nodes, wherein K is less than or equal to N. A set of K entity-specific entry vectors corresponding to the K entity-specific entry nodes are identified. A training vectors is generated by aggregating each of the K entity-specific entry vectors. Thereafter, a machine learning model can be trained using the training vectors and the known classifications.

In another illustrative embodiment, the machine learning model can be used to classify biological entities. As part of the classification, database data comprising N entry nodes to a graph database that includes M nodes is stored, wherein M is greater than N. An entity record, including a plurality of fields, is received. A set of the plurality of fields of the entity record that each matches one of the N entry nodes are identified, thereby identifying K entity-specific entry nodes, wherein K is less than or equal to N. From a plurality of entry vectors, K entity-specific entry vectors corresponding to the K entity-specific entry nodes are identified, wherein each of the plurality of entry vectors includes M entry values, and wherein each of the M entry values for an entry vector represents an importance of a corresponding node to an entry node for the entry vector. An entity signature vector is generated by aggregating the K entity-specific entry vectors. The entity signature vector is then input into the machine learning model. Thereafter, the entity classification for the entity record is received as an output from the machine learning model.

These and other embodiments of the disclosure are described in detail below. For example, other embodiments are directed to systems, devices, and computer readable media associated with methods described herein.

A better understanding of the nature and advantages of embodiments of the present disclosure may be gained with reference to the following detailed description and the accompanying drawings.

### BRIEF DESCRIPTION OF THE DRAWINGS

FIG. 1 shows an example of a graph database with various nodes of different types connected by various edges of different types according to embodiments of the present disclosure.

FIG. 2 is a simplified diagram of a graph database according to an embodiment.

FIG. 3 is a chart showing the source and number of node types according to an embodiment.

FIG. 4 is a chart showing the source and number of edge types according to an embodiment

FIG. 5 shows identification of SEPs from a comparison of health records and SPOKE nodes, and the creation of PSEVs from the identified SEPs according to embodiments of the present disclosure.

FIG. 6 shows the creation of SPOKEsigs by summing PSEVs associated with SEPs found in an individual's EHRs according to embodiments of the present disclosure.

FIG. 7 shows a flowchart illustrating a method for generating a machine learning model using a graph database according to embodiments of the present disclosure.

FIG. 8 shows a flowchart illustrating a method for categorizing entity records using a machine learning model according to embodiments of the present disclosure.

FIG. 9 shows a timeline comparing a cohort of MS patients and a cohort of non-MS patients.

FIG. 10 shows a block diagram for usage of a machine learning model according to embodiments of the present disclosure.

FIG. 11A shows an example machine learning model of a neural network. FIG. 11B shows an example machine learning model of a support vector machine (SVM).

FIG. 12 shows a block diagram of a computer system according to embodiments of the present disclosure.

### DETAILED DESCRIPTION

Current biomedical databases are compartmentalized and lack the connections required to fully model complex biomedical processes.

The Scalable Precision Medicine Oriented Knowledge Engine (SPOKE) is a graph database and contains millions of biomedical concepts with multiple connections among the data. But it is not sufficient that a database contains all necessary data with appropriate connections, in order to classify a new entity (e.g., a patient or drug). While a few biomedical graph databases exist (e.g., SPOKE) their utilization to infer novel relationships and to aid clinicians in patient decision-making has not been yet implemented in any meaningful manner. When applied to biomedical problems, classic machine learning techniques do not account for biological accuracy potentially leading to inaccuracies and instabilities in training and testing a given model.

Using a graph database can provide such relationships, making the model not only statistically powerful but also biologically accurate. Embodiments of the present disclosure can use a large body of EHR data (ideally from millions of patients) and includes patients with various conditions, thus enabling the creation of population control cohorts for any given condition. In addition to the statistical power afforded by the millions of datapoints available, a decision support system based on embedding of individual records onto a biomedical knowledge graph can result in biologically plausible outcomes. Although examples refer where the graph database is SPOKE, not all implementations use SPOKE or require all of the features of SPOKE.

### I. Database Structure

SPOKE is an example graph database that can be used in embodiments of the present disclosure. SPOKE integrates more than 30 popular databases for the biomedical research community, including the Genome Wide Association Studies (GWAS) Catalog (genetics of common diseases), Diseases (disease symptoms), ChEMBL database from the European Molecular Biology Laboratory (EMBL) (chemical compounds), and Search Tool for the Retrieval of Interacting Genes/Proteins (STRING) (protein interactions). Currently, SPOKE contains more than 2 million concepts (nodes) of 12 different types, connected by more than 16 million relationships (edges) of 35 different types. The databases contained in SPOKE involve several disciplines and span several orders of magnitude in space and time, thus allowing complex queries to be made that otherwise would require significant data pre-processing of be impossible altogether.

Graph elements (nodes) in SPOKE are linked to one another through edges that represent the node's relationships in the corresponding database (e.g. the gene IL2Ra is related to the disease multiple sclerosis by the link “associates”, —a relationship pulled from the GWAS catalog). When an element is present in more than one database, additional links are created to relate this element with all its provenances (e.g. the disease multiple sclerosis affects the brain and spinal cord, two tissues from the Diseases database). To facilitate merging of the same concept from different databases, ontologies (e.g. Disease Ontology) and data vocabularies (e.g. medical subject headings-MeSH) are used. All component databases are updated weekly.

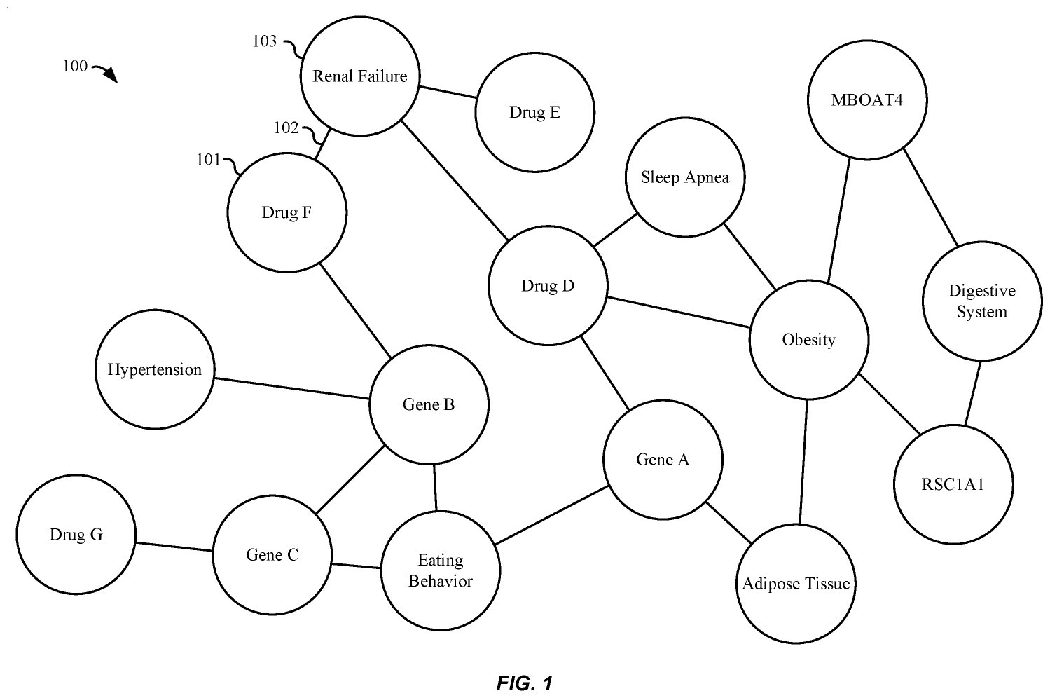

FIG. 1 shows an example of a graph database according to embodiments of the present disclosure. As examples, the graph database can include at least 10,000, 50,000, 100,000, or 400,000 nodes and include at least 100,000, 500,000, 1,000,000, or 10,000,000 edges. FIG. 1 depicts nodes in the graph database and the relationships (edges) between those nodes. For example, a node 101 for drug F is connected by an edge 102 to a node 103 for renal failure. The edge connecting drug F and the node for renal failure show that drug F is associated with renal failure. The edge could encode a variety of relationship types. For instance, the edge 102 could demonstrate that drug F either treats renal failure or causes renal failure. Moving across nodes and edges can uncover previously unknown relationships. For example, there may be a relationship between drug F and drug E because the nodes for each are connected by edges to the node for renal failure.

The graph database (e.g. SPOKE) can be created by merging databases from multiple biomedical sub-disciplines into one comprehensive graph database. By tracing a path across multiple edges and nodes, new pathways incorporating knowledge from multiple sub-disciplines can be uncovered. The types of nodes and the types of edges in the graph databases can vary and depend on the purpose of the database (e.g., for drugs, diseases, etc.) and can depend on the underlying data from which the graph database was generated.

### A. Heterogeneous Networks

Graph databases described herein can use a general framework for representing heterogeneous networks. Heterogeneous networks can include nodes connected by edges with an additional meta layer that defines type. Node type can signify the kind of entity encoded, and edge type can signify the kind of relationship encoded. As examples, edge types can comprise a source node type, a target node type, a kind (to differentiate between multiple edge types connecting the same node types), and a direction (allowing for both directed and undirected edge types). The user can define these types and the user can annotate nodes and edges with the edge or node's corresponding type. The meta layer can be represented as a metagraph consisting of node types (e.g., metanodes) connected by edge types (e.g., metaedges). Metagraphs can include metanodes connected by metaedges. In a heterogeneous network, each path, a series of edges with common intermediary nodes, can correspond to a metapath representing the type of path. A path's meta-path is the series of metaedges corresponding to that path's edges. The possible metapaths within a heterogeneous network can be enumerated by traversing the metagraph.

### B. Graph Database Construction

The included entries in the graph database (e.g., the metaedges and metanodes composing the graph database) can be selected empirically based on a balance among the following properties: 1) quality-relevance to human pathogenesis; high accuracy and an optimal trade-off between false positives and false negatives; 2) reusability-easily retrievable and parsable; mapped to controlled vocabularies; well documented; amenable to reproducible (scripted) analysis; free of prohibitive reuse stipulations; 3) throughput-broad domain-specific coverage generated using systematic platforms that minimize bias; 4) diversified, multiscale portrayal of biology-capturing, in aggregate, many aspects of pathophysiology across multiple levels of biological complexity (e.g., genome, transcriptome, proteome, interactome, metabolome, cell and tissue organization, phenome, etc.). A diversified multiscale portrayal of biology can be achieved in a graph database by integrating information from different biological subdisciplines into the graph. For instance, integrating databases containing information about genes, organs, and diseases can allow for a portrayal of multiple sclerosis that shows how genes acting on leukocytes can impact the disease's presentation. Balancing these considerations, numerous resources can be integrated within computational runtime constraints.

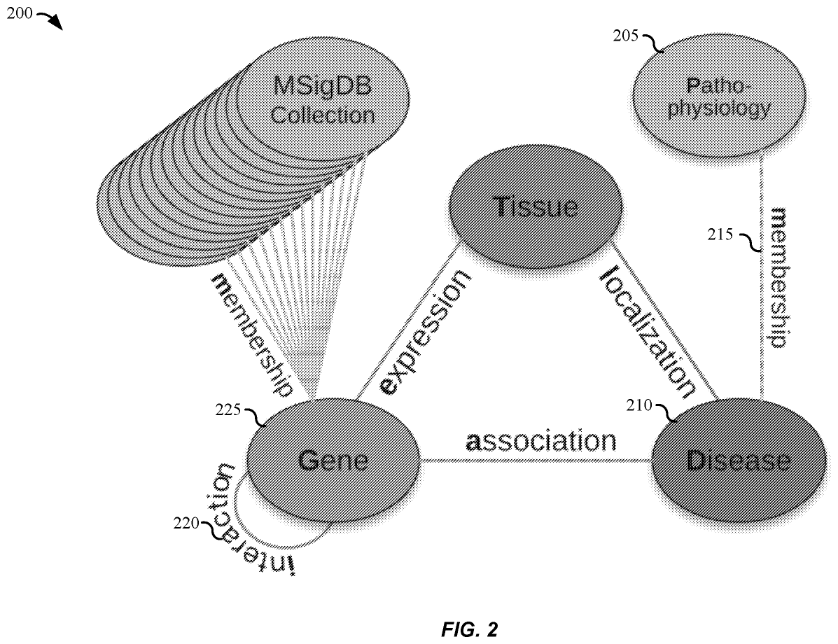

FIG. 2 is a simplified diagram of a graph database 200 according to an embodiment. The graph database 200 can be constructed with one or more annotated node types (e.g., metanodes) and one or more annotated edge types (e.g., metaedges). Tissue 205 is a node type and it is connected to the disease 210 node type by the membership 215 edge type. Edges can connect nodes within a node type. For instance, the interaction 220 edge type can connect nodes within the gene 225 node type.

### 1. Nodes

Nodes in the graph database can be created from entries in biomedical databases. The entries can be selected for including in the graph database using the four criteria disclosed above. For instance, protein-coding genes can be extracted from the Human Genome Organization Gene Nomenclature Committee (HGNC) database. Resources were mapped to HGNC terms via gene symbol (ambiguous symbols were resolved in the order: approved, previous, synonyms) or Entrez identifiers. Disease nodes can be taken from the Disease Ontology (DO). Schriml L M, Arze C, Nadendla S, Chang Y W, Mazaitis M, Felix V, et al. (2012) Disease Ontology: a backbone for disease semantic integration. Nucleic Acids Res 40: D940-946. doi: 10.1093/nar/gkr972 PMID: 22080554.

Relevant disease references can be manually mapped to the DO. For instance, tissues were taken from the BRENDA Tissue Ontology (BTO). Gremse M, Chang A, Schomburg I, Grote A, Scheer M, Ebeling C, et al. (2011) The BRENDA Tissue Ontology (BTO): the first all-integrating ontology of all organisms for enzyme sources. Nucleic Acids Res 39: D507-513. doi: 10.1093/nar/gkq968 PMID: 21030441. Tissues with profiled expression can be included enabling manual mapping. Nodes can be directly imported from the Molecular Signature Database version 4.0. Liberzon A, Subramanian A, Pinchback R, Thorvaldsdottir H, Tamayo P, Mesirov J P (2011) Molecular signatures database (MSigDB) 3.0. Bioinformatics 27: 1739-1740. doi: 10.1093/bioinformatics/btr260 PMID: 21546393. Diseases were classified manually into 10 categories according to pathophysiology.

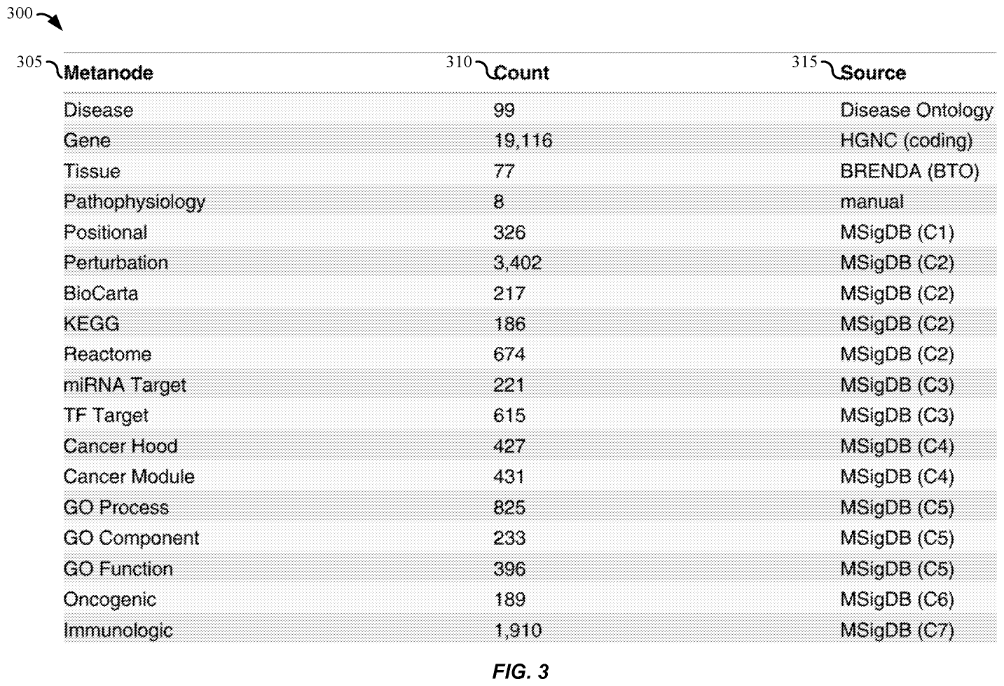

FIG. 3 is a chart 300 showing the source and number of node types according to an embodiment. Edge types are listed in a first column 305, the number of edges for each edge type are listed in a second column 310, and the source for the edges in the edge types is listed in a third column 315.

### 2. Edges

Edges can be connections between nodes and edges can be annotated to show relationships between nodes. Edge can include disease-gene associations, protein interactions, tissue-specific gene expression, disease localization, etc. Edges that are included in the graph database can be selected using the four criteria disclosed above. Disease-gene associations can be extracted from the GWAS Catalog, a compilation of GWAS associations where p<10⁻⁵. Associations can be segregated by disease. GWAS Catalog phenotypes can be converted to Experimental Factor Ontology (EFO) terms using mappings produced by the European Bioinformatics Institute. Associations mapping to multiple EFO terms can be excluded to eliminate cross-phenotype studies. EFO terms can be manually mapped to DO terms and a DO term can be mapped with the term's associations.

Physical protein-protein interactions can be taken from a protein database. For instance. Protein-protein interactions can be taken from iRefIndex 12.0 a compilation of 15 primary interaction databases. iRefIndex can be processed using software (e.g., ppiTrim) to convert proteins to genes, remove protein complexes, and condense duplicated entries.

Tissue-specific gene expression levels can be taken from a gene expression database. For instance, the Genomics Institute of the Novartis Research Foundation (GNF) Gene Expression Atlas. The database entries can be processed before the entries are used to create edges. For instance, starting with GCRMA-normalized and multisample-averaged expression values, 44,775 probes were converted to 16,466 HGNC genes and 84 tissues were manually mapped and converted to 77 BTO terms.

Edges values can be determined by examining literature co-occurrence. For instance, literature co-occurrence was used to assess whether a tissue is affected by a disease. A text mining system, such as CoPub 5.0, can be used to extract statistics about the co-occurrence of terms in literature. As an example, CoPub 5.0 can be used to extract R-scaled scores between tissues and diseases measuring whether two terms occurred together in Medline abstracts more than would be expected by chance. The R-scaled score can be used as a threshold criteria for edge inclusion.

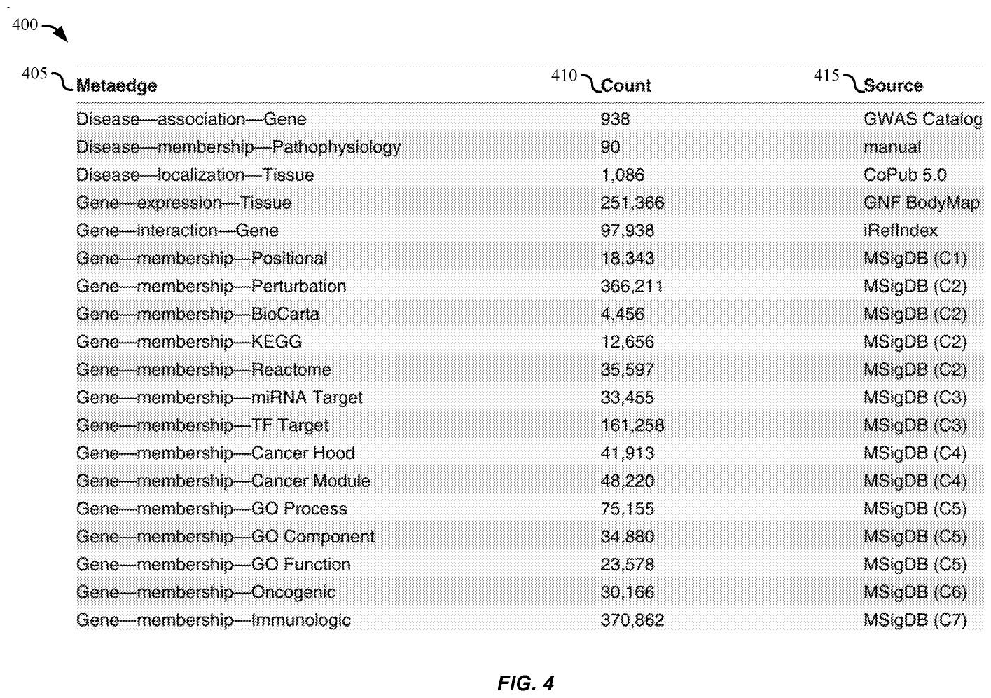

FIG. 4 is a chart 400 showing the source and number of edge types according to an embodiment. Node types are listed in a first column 405, the number of nodes for each node type are listed in a second column 410, and the source for the nodes in the node types is listed in a third column 415.

### 3. Feature Computation Metrics

Features can be characteristics of entries in a database. Edges and nodes can be annotated with features and the features can be independent variables for a machine learning model. Features length and number of connections between in a graph database can allow a model to make predictions about the relationship between nodes. The features discussed in this section, path count (PC) and degree weighted path count (DWPC), can be weights for an edge connecting two nodes.

Path count (PC) can be a feature representing the number of paths between a source and target node. However, PC may not adjust for the extent of graph connectivity along the path. Paths traversing high-degree nodes can account for a large portion of the PC. Normalized path count (NPC) is a metric that includes a PC denominator to adjust for connectivity. The denominator for NPC equals the number of paths from the source to any target plus the number of paths from any target to the source.

$${NPC}_{m} ( s , t ) = \frac{{PC}_{m} ( s , t )}{{\sum }_{{t}_{i} ∈ {T}_{m}} {PC}_{m} ( s , t ) + {\sum }_{{s}_{i} ∈ {S}_{m}} {PC}_{m} ( s , t )}$$

Where, m is the metapath, s is the source node, t is the target node, Sm is the set of nodes corresponding to the source node of m, and Tm is the set of nodes corresponding to the target node of m. We adopt the any source/target concept to compute the two GaD features. GaD can be the predicate of the edges connecting genes and diseases (e.g., a weight representing an association between the gene and disease). However, when dividing the PC by a denominator each path composing the PC can receive a distinct degree adjustment. If two paths-one traversing only high-degree nodes and one traversing only low-degree nodes-compose the PC, the network surrounding the high-degree path can monopolize the NPC denominator and overwhelm the contribution of the low-degree path despite its specificity. This problem can be mitigated by the degree-weighted path count (DWPC) which can individually down weight paths between a source and target node. The paths can receive a path-degree product (PDP) calculated by: 1) extracting all metaedge-specific degrees along the path (Dpath), where each edge composing the path contributes two degrees; 2) raising each degree to the −w power, where w≥0 and is called the damping exponent; 3) multiplying all exponentiated degrees to yield the PDP.

$$PDP ( path ) = {\prod }_{d ∈ {D}_{path}} {d}^{- w}$$

The DWPC equals the sum of two PDPs.

$$PDP ( s , t ) = {\prod }_{path ∈ {Paths}_{m} ( s , t )} PDP ( path )$$

II. Extracting Relevant Information from the Database

Data leads to information and information leads to knowledge. Vast amounts of data are being produced at an incredible pace (from 33×1021-zeta-bytes in 2018 to a predicted 175 ZB by 2025) and this rapid explosion in the generation of data is causing databases and repositories to exponentially increase in size. However, the rate at which we transform data into (medical) knowledge has not improved accordingly. This lag could be explained by the difficulty of making logical connections across the vast number and variety of datasets that continue to be created in the biomedical space.

By integrating large amounts of knowledge from multiple sources, the Scalable Precision medicine Oriented Knowledge Engine (SPOKE) aims at facilitating the retrieval of specific and relevant information and even the emergence of new knowledge from it. The high interconnectedness of the concepts represented by the nodes of the knowledge network (genes, proteins, compounds, phenotypes) represent physical or functional (effective) relationships that are part of one system that functions as a whole. Thus, the biomedical knowledge network, if sufficiently comprehensive, can also reflect the actual organization of an organism as a complex dynamical system, and its analysis may facilitate discoveries of a new level of systems knowledge.

By merging knowledge stored in siloed databases we gain knowledge because it allows the traversal of information and facilitates machine learning approaches to be applied in a way that you could not in a federated system. The SPOKE database has a particular utility for users who specialize in one scientific field but lack computational expertise. Such users can navigate the database, starting at their area of expertise, with the confidence that they are exploring well established knowledge that is part of the network.

However, even with such a graph database, the onus is on the user to perform searches and compare to a new data set. To address such a problem, embodiments of the present disclosure can use a graph database to analyze records (including input data and output labels) to transform the dataset to usable training vectors for machine learning. For example, a graph database in combination with a dataset of entity records can be used to generate training vectors to train a machine learning model. The entity records can correspond to a cohort of entities with known classifications (output labels). The cohort can have as few as two known classification (e.g., presence or absence of a disease) but may include more classifications (e.g., when there are different levels of efficacy of a drug). Even though examples are provided with respect to SPOKE, the skilled person will appreciate that other graph databases may be used.

In generating a training vector, embodiments can identify overlapping concepts from the entity's records (EHRs) and the SPOKE graph database. Entry nodes associated with overlapping concepts are known as SPOKE entry points (SEPs) according to an embodiment of the disclosure. A propagated entry vector (PSEV), encoding the importance for each graph database node for a particular SEP (entry node), is created for each SEP.

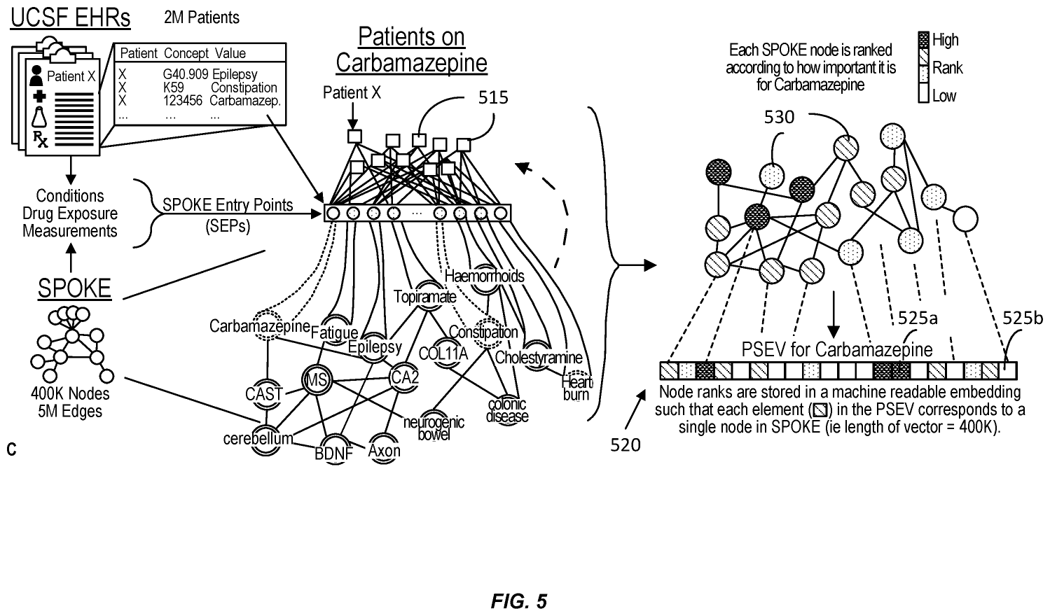

FIG. 5 shows identification of SEPs from a comparison of health records and SPOKE nodes, and the creation of PSEVs from the identified SEPs according to embodiments of the present disclosure. In FIG. 5, the EHRs are combined with SPOKE (e.g., using condition or drug exposure measurements) to identify the SEPs. As shown, a PSEV for Carbamazepine is created using SEPs identified from overlapping concepts found in EHRs for a cohort of patients on Carbamazepine and SPOKE. Certain nodes are shown as corresponds to SEPs, e.g., Carbamazepine, fatigue, epilepsy, Topiramate, constipation, hemorrhoids, Cholestyramine, and heart burn. These are the nodes that exist in both the EHRs and the graph database.

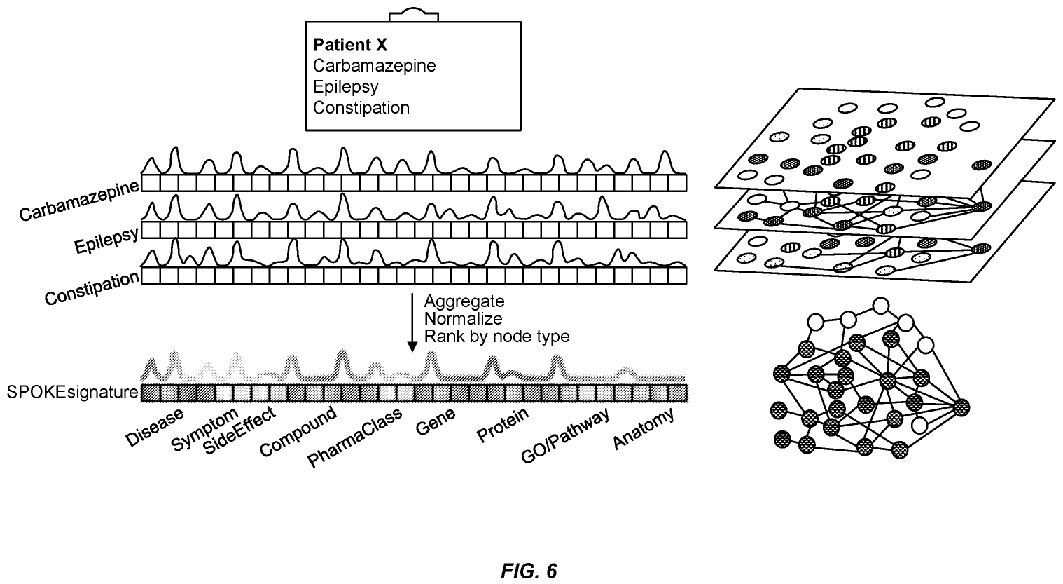

A process is then used to determine how important a node is for each SEP, thereby generating PSEVs. PSEV generation is discussed in section III.D below. A training vector (SPOKEsig) can be created for each cohort member by summing each PSEV associated with the SEPs found in that patient's EHRs. SPOKEsig creation is described later in FIG. 6 where PSEVs for carbamazepine, epilepsy, and constipation are aggregated to create a SPOKEsig for patient X. SPOKEsig creation is described in section III.E below.

The machine learning model can be trained using the SPOKEsigs. The intended output from the machine learning model, a classification, is known for the cohort, so the model can be trained, using these vectors, to output the known classification. The training process involves inputting the vectors into the machine learning model and observing the output. The machine learning model is trained, by changing model variables, until the machine learning model outputs the correct classification for a given input. Training the model can involve creating training vectors from entity records with known classifications and feeding the training vectors into a learning modules as part of a training process.

After the machine learning model is trained, the model can be used to classify new biological entities. To classify a new entity, an entity-specific vector is generated and input into the trained machine learning model. The entity signature vector generation process is similar to SPOKEsig creation except the entity signature vectors are made for individuals with an unknown classification. PSEVs are identified for each SEP in the unclassified entity's EHRs and summed to produce an entity-specific vector. The entity-specific vector is input into the trained machine learning model and a classification for that entity is output. An entity signature vector can be generated for a new entity record and entered into the model to generate an entity classification. In this way an unknown biological entity can be classified using a machine learning model.

### III. Training a Model for Classifying Biological Entities

In order to convert structured EHR data within these individual snapshots into SPOKE embeddings, the population level interactions between different EHR concepts (e.g., fields) and SPOKE may be established. This is achieved through PSEVs which are machine-readable embeddings that quantify the significance of each node in SPOKE for a given cohort of patients. PSEVs are generated using a modified version of topic specific page rank to learn and embed the importance of each node in SPOKE for a given restart node or set of nodes. These restart nodes, called SPOKE Entry Points (SEPs), are any concept in the input data that overlaps with a node(s) in SPOKE.

Connecting EHRs to SPOKE provides real-world context to the network thus enabling the creation of biologically and medically meaningful “barcodes” (i.e., embeddings) for each medical variable that maps onto SPOKE. These barcodes can be used to recover purposely hidden network relationships such as Disease-Gene, Disease-Disease, Compound-Gene, and Compound-Compound. Embedding EHR concepts into SPOKE involves identifying overlapping points between the EHRs and SPOKE and populating these SEPs through the KG using a modified page rank algorithm to create a unique Propagated SPOKE Entry Vectors (PSEV).

### A. Store Database Structure

SPOKE specifies the types of nodes and the types of connections between certain nodes based on biologically meaningful relationships based on expert knowledge of its curators.

To create a database with sufficient breadth to categorize new relationships or patient outcomes, the graph database incorporates data from over 30 biomedical databases. Each biomedical database is incorporated into the graph database by identifying nodes in both the biomedical database and the graph database that describe the same concept (e.g., node for multiple sclerosis linked to the gene IL2Ra from a genetic database and a node for multiple sclerosis linked to brain and spinal cord from a disease database). The nodes that describe the same concept are consolidated into one final graph database node. Edges (relationships) between nodes are preserved during this consolidation (e.g., final node for multiple sclerosis linked to IL2Ra, brain, and spinal cord). A graph database according to embodiments of the present disclosure is shown in FIG. 1 with nodes and edges connecting the nodes. In this way, comprehensive relationship neighborhoods are created for millions of concepts and integrated into a single knowledge graph (i.e. graph database).

### B. Receive EHR Data Including, Identifying EHR Fields

Patient Electronic Health Record (“EHR”) data is received. The EHR data includes standardized labels corresponding to the classifications of the patients (e.g., a disease diagnosis, a lab result, a therapeutic drug, etc.). Select structured EHR data tables contain codes, referred to as EHR fields, that can be linked to standardized medical terminology (e.g., standardized terminology). Specifically, EHR fields can be diagnostic codes (ICD9CM or ICD10CM), medication order codes (translated to RxNorm), or lab codes (LOINC). EHRs can contain actual measurements from test orders or judgements from medical professionals about whether the results were normal or abnormal. EHRs can be de-identified to protect patient privacy, and EHR fields can be associated with an anonymous patient identifier (ID) or an encounter identifier (ID). A patient ID can be used to group EHR fields by patient and an encounter ID can be used to group EHR fields for individual encounters (e.g., an appointment with a doctor, a hospital stay, etc.).

FIG. 5 depicts a series of EHR (entity) records affiliated with entity Patient X, as well as other patients 515. The records in FIG. 5 are shown containing both codes, under the concept column, and plain language descriptions of those codes, under the value column. Certain EHR fields can allow for patients to be classified, using them as input in a machine learning model.

However, just using the EHR fields directly does not incorporate the knowledge of the relationships among the fields and the resulting model only represents a purely statistical process. Using a graph database can provide such relationships, making the model not only statistically powerful but also biologically accurate.

This approach can use a large body of EHR data (ideally from millions of patients) and include patients with various classifications, e.g., with a disease and without a disease thus enabling the creation of population control cohorts for any given condition.

Standardized EHR (entity record) fields allows information from individual patient records to be connected to the SPOKE graph database. Identifying and standardizing EHR fields facilitates SEP (entry node) identification discussed below in section C “Identifying SEPs.”

### C. Identifying SEPs

Select structured data tables from the EHR are used to identify EHR concepts that can be directly linked a node in SPOKE. These points of overlap between the EHRs and SPOKE are called SPOKE Entry Points (SEPs). The data tables are then used to create 3,233 PSEVs, one for each identified SEP. Each structured EHR table contains codes, referred to as EHR concepts, that can be linked to standardized medical terminology (e.g., standardized terminology, medical concepts, concepts, nodes, edges, etc.). EHR concepts can be diagnostic codes (ICD9CM or ICD10CM), medication order codes (translated to RxNorm), or lab codes (LOINC). Although 3,233 represents a sizable proportion (7.5%) of nodes in SPOKE, most nodes are not directly reachable, thus potentially diluting the power of the network's internal connectivity. To address this challenge, a modified version of the random walk algorithm was used to propagate all 3,233 SEPs through the entirety of the knowledge network, thus creating a unique PSEV (i.e. medical profile) for each of the selected clinical features in the EHRs.

Once the EHR fields are identified, data from EHRs and SPOKE can be linked. This process can use additional databases, such as the Unified Medical Language System (UMLS) to map EHRs to SPOKE. UMLS is a medical terminology database that contains a list of medical concepts (e.g., concepts) where each concept is linked to one or more terms (e.g., codes, synonyms, etc.) for the concept. Such a terminology database can group (link) together terms that all correspond to a same concept, e.g., a same disease or condition. For example, these additional databases can be used to connect concepts in the EHRs to nodes in SPOKE. The Logical Observation Identifiers Names and Codes (LOINC) is a database that contains a list of laboratory tests that are linked to analytes. MarkerDB is a database that contains lists of biomarkers that are linked to a list of diseases.

FIG. 5 shows arrows pointing from a set of EHRs and SPOKE indicating a comparison between entity fields (EHRs) and graph database (SPOKE) nodes to identify overlapping conditions, drug exposures, and measurements. EHR fields and SPOKE nodes are compared to identify which fields and nodes correspond to the same data type. The points of overlap between the EHRs and SPOKE are recorded as SPOKE entry points (“SEPs”). SEPs allow the graph database to be used for real world predictions because they are concepts that are both observable in a clinical setting and part of the graph database. In FIG. 5, entry nodes (SEPs) are identified for the overlapping conditions, drug exposures, and measurements.

SEP (entry node) identification allow EHR (entity) data to be linked to the SPOKE graph database. SEPs facilitate uncovering relationships between SPOKE nodes and EHR fields because SEPs represent a point of overlap between the records and database.

### D. Generate PSEVs (“Enrichment” of EHRs Based on the Graph Database)

PSEVs are generated in batch for an entire population (e.g. the UC Health System, Kaiser, or Providence), usually millions of patients. A PSEV (e.g., an entry vector, and entity-specific entry vector, etc.) can be created for any EHR variable(s) with a cohort of patients. Some examples of simple cohort selections are: patients with a given disease, patients prescribed a certain medication, patients with an abnormal lab test, or patients of a specific gender. However, cohorts can be as complex as the user desires. A complex cohort could be patients with multiple sclerosis age 50-60 taking Ocrevus. Patients can be excluded from a cohort if the total number of EHR variables or SEPs in the patient's records is below a threshold. For instance, a patient may be excluded from a cohort unless the patient's records contain at least three SEPs, or a patient cohort may require that patients in the cohort show five separate diagnosis's for multiple sclerosis (MS) (e.g., five separate MS diagnosis codes in the patient's EHRs).

For each cohort (usually thousands), a Propagated SPOKE Entry Vector (“PSEV”) can be created using a process similar to physical diffusion (e.g., smoke in a closed environment, dye in a water container, etc) whereby each SEP is extended to their neighbors in the graph with a certain probability. Each resulting PSEV has exactly a length equal to the number of nodes in SPOKE, with each value in the PSEV corresponding to the relevance of a particular node in SPOKE for a given set of SEPs for the selected patient cohort. In other words, each value in a PSEV reflects the importance of the corresponding node in the database for that cohort. The importance of a given SEP can be the proportion of patients in a cohort that have an EHR field that is mapped (e.g., linked) to the SEP. Thus, if there are N cohorts in the entire population, and the graph database has M nodes, there will be N vectors (PSEVs), each of length M. PSEVs represent the relevance of the information found in EHRs given all known basic science data that is not achievable in a clinical setting.

In FIG. 5, PSEV 520 is shown with vector values 525 corresponding to nodes in the graph, with correspondence indicated by dotted lines. The nodes 530 and corresponding vector clause 525a-b are shaded a color gradient according to their importance for the SEP carbamazepine. The color gradient ranges from dark grey for important nodes 525a to white for unimportant nodes 525b. An arrow pointing from graph database to an array of connected squares labeled “PSEV for Carbamazepine” depicts PSEV creation using the graph database. The squares (vector values 525a-b) in the PSEV are shaded from grey to white like the graph database nodes to represent that the PSEV encodes the importance of each node for a given SEO. Once a patient cohort has been selected, the PSEV is created using the following steps for each PSEV.

First, a SEP transition vector of length N (where N=the number of SEPs) is created and every value is set to zero. Then, for each patient in a cohort, a binary vector of all of the SEPs (indicating whether the patient in question has a given SEP or not) in the patient's EHRs is created, and each value in the binary vector is then divided by its sum to normalize the vector. This normalized patient vector is then added to the SEP transition vector. Once every patient is accounted for, the SEP transition vector and divided by the sum. The final SEP transition vector represents how important each SEP is for the current patient cohort.

Second, an adjacency matrix is created using the edges in SPOKE to initialize the SPOKE transition probability matrix (TPM) in which each column sums to 1. The adjacency matrix is an square matrix with a column for each node and a row for each node. The adjacency matrix is initialized with all values set to 0, but if two nodes are connected, that intersection will be filled with a 1. The SPOKE TPM is then multiplied by 1-β where β equals the probability of random jump. B can have a value between 0-1 and the β value may be different for each cohort. Then the probability of randomly jumping to one of the SEPs is incorporated. This is achieved by multiplying the SEP transition vector by β and adding the value of each SEP to the corresponding row in the TPM. Following this, the columns of the SPOKE TPM will again sum to one.

The PSEVs can then be generated using a modified version of the PageRank algorithm. In this version of PageRank, for each PSEV, the random walker starts at the corresponding SEP and traverses the edges of SPOKE until randomly jumping out of SPOKE (at probability β). The walker will then enter back into SPOKE through any SEP using the probabilities found in the corresponding column of the SPOKE TPM. The vector can be updated in a step-wise fashion once per cycle. The walker will continue this cycle until the difference between the rank vector in the current cycle and the previous cycle is less than or equal to a threshold (a). The final rank vector is the PSEV and contains a value for every node in SPOKE that is equivalent to the amount of time the walker spent on each given node.

PSEVs (propagated entry vectors) encode each SPOKE node's importance for a particular patient cohort. PSEVs are an example of propagated entry vectors according to the present disclosure. PSEVs are also used to generate the training vectors (SPOKEsigs) in the process described below in section E “Generating patient specific training vectors.”

### E. Generating Patient Specific Training Vectors

The PSEVs provide a representation of the embedding of millions of EHRs onto SPOKE. The PSEVs can be used as building blocks to “enrich” an individual EHR into a feature vector that is used for training the machine learning model. Such an input feature vector includes the data from millions of EHR as well as the connections of the graph database. The individual input feature vectors (e.g., Patient Specific Profile Vectors, patient specific training vectors, etc.) are referred to as SPOKEsigs. SPOKEsigs can be used to train a machine learning model for an individual entity or patient. For example, a model trained using PSEVs or SEPs can be used to determine if a drug is a likely treatment for a disease. A SPOKEsig can be used to train a model to determine if a drug is a likely treatment for a disease as the disease has presented in an individual patient. Accordingly, SPOKEsigs can allow models to be trained to make classifications for individual entities.

In essence, SPOKEsigs are vectors that can be created for a specific patient. Like a PSEV, each SPOKEsig has a length of the number of nodes in SPOKE, with each value in the SPOKEsig corresponding to a particular node in SPOKE. Each value in the SPOKEsig is the importance of the corresponding node in the database for that particular patient's current EHR. The nodes can be ranked (e.g., from 1 to the number of nodes in SPOKE) and the most important node can be equal to the number of nodes in SPOKE.

FIG. 6 depicts training vector (SPOKEsig) generation according to embodiments of the present disclosure. In this example, an entity (Patient X) has entity record (EHR) fields for the disease epilepsy, the symptom constipation, and for taking the drug Carbamazepine. The three entity record fields correspond to graph database nodes so they are also entry nodes with associated propagated entry vectors. In order to generate a training vector (SPOKEsig), the three propagated entry vectors (PSEVs) corresponding to the fields found in the patient's entity records (EHRs) are summed (e.g., aggregated) to create a training vector (SPOKEsig) for that particular patient. The PSEVs can be aggregated by vector addition to produce a vector sum. The corresponding PSEVs can be for fields that are positive or present for that given patient. The SPOKEsig eventually can be used as an input to train a machine learning model.

A SPOKEsig can also be created for a limited portion of a patient's medical history. Instead of selecting every SEP associated with a patient, only the SEPs from EHRs that were generated over a given time period can be chosen. SEPs from EHRs that were generated by certain types of medical visits can also be selected. For instance, SEPs generated by visits to endocrinologists and gynecologists can be chosen while SEPs from cardiologist visits can be excluded. The PSEVs that correspond to the limited set of SEPs are then summed to create a new SPOKEsig for that period of time. Using this method, training vectors can be created for patients at various stages in their illness (e.g. SPOKEsig for a multiple sclerosis patient 3-5 years before their diagnosis).

Patients in a first cohort can (e.g., patients with a MS diagnosis code in their EHRs) can be aligned with patients in a second cohort (e.g., patients without a MS diagnosis code in their EHRs). The cohorts may be filtered to exclude patients based on the length of the patients medical history. For instance, the first cohort may be filtered to exclude patients who do not have at least seven years of medical records (e.g., EHRs) prior to a MS diagnosis. The second cohort can be limited to patients, not diagnosed with MS, who have at least seven years of EHR data. Patients may be excluded from a cohort based on the patient's treatment history. A patient could be excluded from the first cohort if the patient's EHRs indicate that the patient received a treatment that can impact MS prior to the patient's diagnosis for MS.

SPOKEsigs (training vectors) encode each SPOKE node's importance for a particular patient over a given time period. SPOKEsigs are an example of training vectors according to the present disclosure, and SPOKEsigs are inputs used to train the machine learning model.

### IV. Methods

The methods include techniques for training a model and a method for using the model to classify entities. Section A “Method for training a model for classifying biological entities” relates how to create training vectors (e.g., SPOKEsigs) and how to use the vectors to train a machine learning model. Section B “Method for classifying biological entities using a machine learning model” relates how to create input vectors (e.g., SPOKEsigs, entity signature vectors, etc.) for an entity and how to use the vectors, and a trained model, to receive a classification for the entity.

### A. Method for Training a Model for Classifying Biological Entities

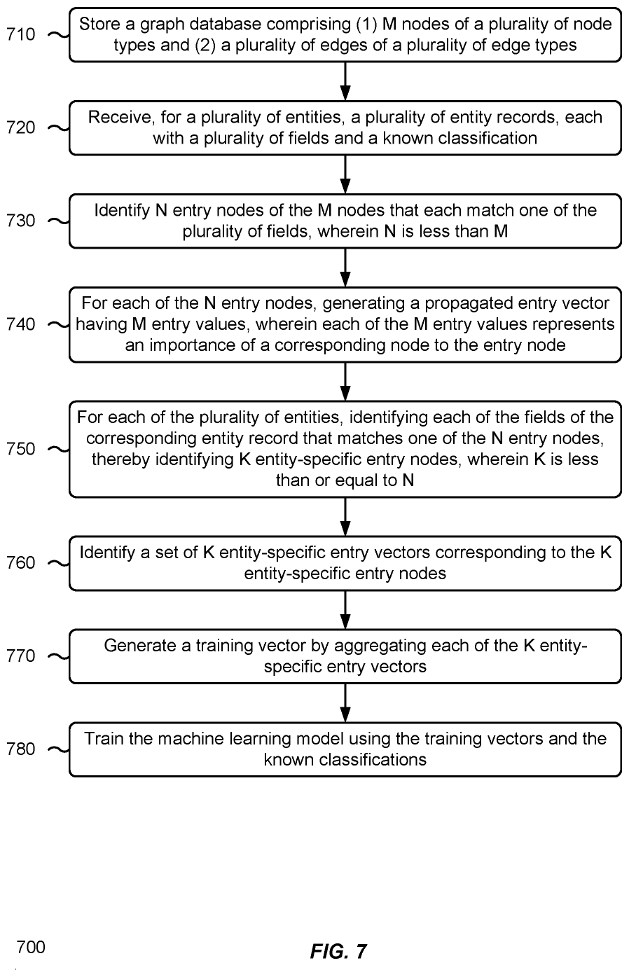

FIG. 7 is a flowchart of an example process 700 for training a machine learning model using input training vectors transformed using a graph database according to embodiments of the present disclosure. Process 700 can use a graph database (e.g., SPOKE) to obtain training vectors for training a machine learning model. The graph database can be used to filter out key information from the input data records, which helps the model be trained on only relevant information. Further, the data of a given entity (e.g., a patient) can be transformed using propagated entry vectors that were generated using the entity records and the structure of the graph database. Such transformations allow effective training of the model, increasing efficiency and stability of the model. Process 700 may be performed by a computer system, which may take carious forms, e.g., a server computer, a laptop computer, a mobile device, etc.

At block 710, a graph database is stored. The graph database can comprise (1) M nodes of a plurality of node types and (2) a plurality of edges of a plurality of edge types. Node types can include nodes for genes, proteins, tissues, pathophysiologies, pathways, perturbation signatures, motifs, chemical compounds, genotypes, phenotypes, etc. Edge types can include gene-disease association, disease pathophysiology, gene localization, tissue-specific gene expression, protein interaction, gene set membership, etc. The different node types, edge types, or EHR fields can be standard terminology in a technical field such as biology or medicine. For example, such terms can be parts of a cell, parts of a body, measured symptoms or classifications, genetic properties, demographic information, and the like. Such terms can also be referred as concepts in the technical field. SPOKE is an example of such a graph database, as described herein, including in the appendices.

A new graph can be added to the graph database. The new graph being added to the graph database can be generated from a new database with nodes (e.g., database nodes) connected by edges (e.g., database edges). The nodes in the new graph, as well as the graph database, may refer to standardized terminology (e.g., afore-mentioned concepts, standardized medical terminology). Nodes in the new graph and the graph database may refer to the same standardized terminology. The new graph may be added to the graph database by merging nodes from the first graph and the graph database that refer to the same standardized terminology. The nodes may be merged by adding an edge between the merged nodes. The added edge can indicate that the merged nodes refer to the same standardized terminology.

The node types for the new graph (e.g., database node types) can include nodes for genes, proteins, tissues, pathophysiologies, pathways, perturbation signatures, motifs, chemical compounds, genotypes, phenotypes, etc. The edge types for the new graph (e.g., database edge types) can include gene-disease association, disease pathophysiology, gene localization, tissue-specific gene expression, protein interaction, gene set membership, etc.

At block 720, a plurality of entity records, each with a plurality of fields and a known classification, is received for a plurality of entities. For example, the computer system may receive, for a plurality of entities, a plurality of entity records (e.g., EHRs), each with a plurality of fields and a known classification, as described herein. Example entities include patients, drugs, and animal subjects. Entity records can be excluded if the entity records were created outside of a specified time period, and, for example, entity records created two or more years before a MS diagnosis can be excluded. Excluding an entity record can mean that A record source can be an individual, organization, or entity that created the entity record. For instance, “cardiologist” can be the record source for an entity record created during a cardiologist visit. Entity records can be excluded based on the entity records' source (e.g., entity records created by a cardiologist are excluded).

At block 730, N entry nodes of the M nodes that each match one of the plurality of fields are identified, wherein N is less than M. For example, the device may identify N entry nodes (e.g., SEP nodes) of the M nodes that match one of the plurality of fields, wherein Nis less than M, as described herein. A field can match in a variety of ways. For example, the text label for a field can be exactly the same as the text label for a node. In some implementations, some characters can be different but still be within a threshold to match. Synonyms can also be used to match, e.g., a table can list a group of text labels that match to each other.

A node and field can match if the node and field are linked in a terminology database. Terminology databases, such as the UMLS, can contain groupings of concepts (e.g., definitions, medical codes, synonyms, etc.) that are linked together. The terminology database can act as a thesaurus, and the concepts can be linked because an expert curating the database has determined that the concepts are sufficiently related (e.g., synonyms).

The number of fields in an entity's records that refer to the same entry node can be compared to an entry node threshold, and, for example, a patient (e.g., entity) can be selected and used to create a training vector if the number of MS diagnoses (e.g., fields referring to the MS node) in the patient's medical records (e.g., entity records) exceeds entry node threshold. An entity can be selected and used to create a training vector if the total number of fields in the entity's records (e.g., EHRs) exceeds a threshold number of fields (e.g., field threshold). An objective node can be a node that corresponds to a concept that the trained machine learning model is intended to classify. For instance, the objective node can be a MS node for a model that is trained to diagnose patients with MS.

An entity can be selected and used to create a training vector if the number of fields in the entity's entity records corresponding to the objective node exceeds a threshold. For instance, an entity may be selected if the entity's records contain five or more MS diagnoses. An entity can be sorted into one or more cohorts based on whether the entity's records contain a threshold number of fields that correspond to the objective node (e.g., objective threshold). For instance, an entity can be sorted into a first cohort if the entity's records contain one or more MS diagnoses and the entity can be sorted into a second cohort if the entity's records contain zero MS diagnoses. Training vectors can be created from the records in each cohort and a machine learning model can be trained for each cohort.

At block 740, for each of the N entry nodes, a propagated entry vector (e.g., a PSEV) having M entry values is generated, wherein each of the M entry values represents an importance of a corresponding node to the entry node. An entry value can be a number representing the importance of the corresponding node to the entry node where the entry value can be 1 for the least important node and the entry value can be M for the most important node. PSEVs, an example of propagated entry vectors, are discussed in Section III subsection D.

At block 750, for each of the plurality of entities, each of the fields of the corresponding entity record that matches one of the N entry nodes is identified, thereby identifying K entity-specific entry nodes, wherein K is less than or equal to N. A field and node can match in a number of ways, e.g., as described herein. A field and node may both refer to the same standardized terminology such as diagnostic codes (ICD9CM or ICD10CM), medication order codes (translated to RxNorm), or lab codes (LOINC). Alternatively, the node and field can be linked by identifying different codes or descriptions that refer to the same medically relevant concept (e.g., standardized terminology). The node and field can be linked via a database such as the UMLS database if the node and field correspond to terms that are linked to the same standardized terminology. Corresponding entity records for an entity can include EHRs generated for that entity.

At block 760, a set of K entity-specific entry vectors corresponding to the K entity-specific entry nodes is identified. The set of vectors can be stored and associated with the entry node, and thus the K entity-specific entry vectors can be retrieved based on the entry nodes. The set of entity-specific entry vectors may be all vectors associated with all of that entity's record fields or the set may be vectors associated with a subset of the entity's records or a subset of entity record fields. An entity-specific entry vector that corresponds to an entity-specific entry node can be a vector that was generated using the entry node.

At block 770, a training vector (e.g., SPOKEsig) is generated for an entity by aggregating each of the K entity-specific entry vectors associated with that entity's records. As examples, a training vector can be generated using all of that entity's records or it can be made for a subset of the entity's records. Subsets of an entity's records can include records generated over a limited time period, or records generated under specific conditions. The entity-specific entry vectors can be aggregated by summing each of the K entity-specific entry vectors.

At block 780, the machine learning model is trained using the training vectors and the known classifications. Example details on training a machine learning model can be found in section VI. Examples of the machine learning model may include deep learning models, neural networks (e.g., deep learning neural networks), kernel-based regressions, adaptive basis regression or classification, Bayesian methods, ensemble methods, logistic regression and extensions, Gaussian processes, support vector machines (SVMs), a probabilistic model, and a probabilistic graphical model. Embodiments using neural networks can employ using wide and tensorized deep architectures, convolutional layers, dropout, various neural activations, and regularization steps.

Process 700 may include additional implementations, such as any single implementation or any combination of implementations described below and/or in connection with one or more other processes described elsewhere herein.

Although FIG. 7 shows example blocks of process 700, in some implementations, process 700 may include additional blocks, fewer blocks, different blocks, or differently arranged blocks than those depicted in FIG. 7. Additionally, or alternatively, two or more of the blocks of process 100 may be performed in parallel.

### B. Method for Classifying Biological Entities Using a Machine Learning Model

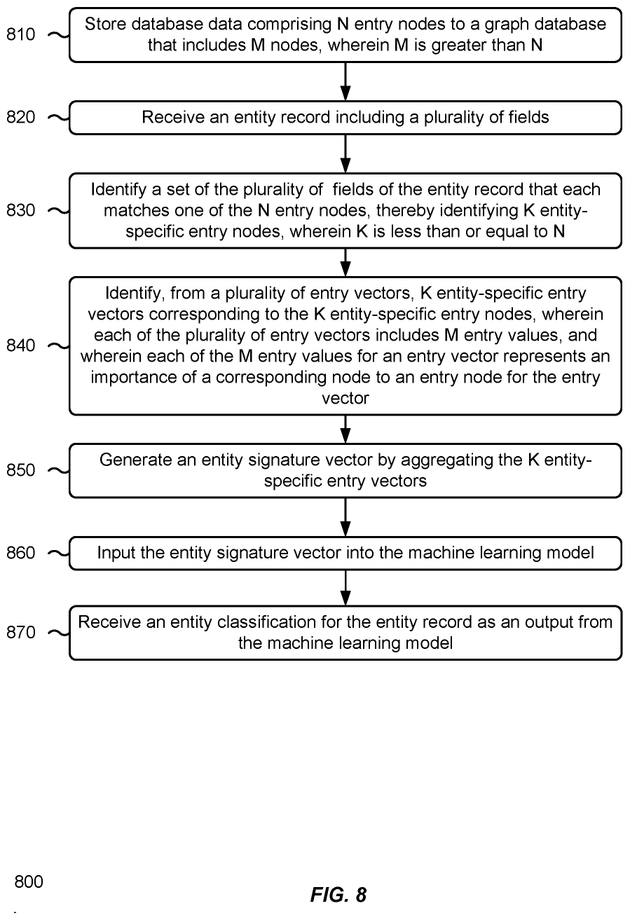

FIG. 8 is a flowchart of an example process 800 for classifying biological entities using a machine learning model according to embodiments of the present disclosure. Process 800 can use a machine learning model trained with training vectors generated by a graph database (e.g. SPOKE) to classify new biological entities. An entity signature vector is generated for each unclassified entity and used as an input to the machine learning model. The machine learning model then outputs a classification for the biological entity associated with the entity signature vector.

At block 810, a database data comprising N entry nodes is stored to the graph database (e.g., SPOKE). The entry nodes can be defined relative to a specific set of entity records, e.g., which were used to train the machine learning model. The graph database may include M nodes, where M is greater than N. Entry nodes can correspond to any medically relevant concept, including diseases, genes, symptoms, or drugs that appear in fields of entity records that are to be classified.

At block 820, an entity record (e.g., EHR) including a plurality of fields is received. Entity records may be records related to medical treatment and entity record fields can include information related to diagnosis and treatment, such as test results, drug prescriptions, symptoms, or diagnoses.

At block 830, a set of the plurality of fields of the entity record that each matches one of the N entry nodes is identified, thereby identifying K entity-specific entry nodes (e.g., SEPs), wherein K is less than or equal to N. The matching be in done in various ways, e.g., as described herein. The entity record can be of a similar type as entity records that were used to train the machine learning model. If this entity record is of a different type, a transformation can be performed on the data to obtain data in a similar format as the entity records used for training.

At block 840, K entity-specific entry vectors corresponding to the K entity-specific entry nodes are identified from a plurality of entry vectors (e.g., PSEVs). Each of the plurality of entry vectors can include M entry values, and each of the M entry values for an entry vector can represent an importance of a corresponding node to an entry node for the entry vector. The entry vectors may be previously generated, while training the machine learning model, or may be created as part of the method for classifying biological entities. The entry vectors can be retrieved, e.g., from a database, where an entry vector is stored in association with the corresponding entry node.

At block 850, an entity signature (e.g., SPOKEsig) vector is generated by aggregating the K entity-specific entry vectors. As examples, an entity signature vector can be generated using all of that entity's records or it can be made for a subset of the entity's records. Subsets of an entity's records can include records generated over a limited time period, or records generated under specific conditions.

At block 860, the entity signature vector is input into the machine learning model. How an input is entered into the machine learning model can depend on the type of machine learning model. Inputting the signature vector into the model may require preprocessing and the vector may be input in its entirety or it may be input in sections.

At block 870, an entity classification for the entity record is received as an output from the machine learning model. Ideally, predicted entity classification corresponds to the true entity classification for new entity signature vector. In some implementations, the entity classification can be provided as a probability for a given classification.

The entity classification can be used to determine a treatment for a patient (e.g., entity). A treatment can be a therapy, drug, surgery, etc. that is used to manage a disease or condition. Treating a patient can mean applying a treatment to the patient. A treatment plan can be one or more treatments used together to manage a disease or condition. An example of a treatment plan is the antiretroviral therapy (ART) “cocktail” that is used to treat an human immunodeficiency virus (HIV) infection. An existing treatment plan can be altered based on the entity classification. An existing treatment plan can be a treatment plan that is currently being used to treat a patient. For instance, an entity classification may indicate that a MS patient is unlikely to respond to a drug in the patient's current treatment plan. The patient's doctors may swap the drug for an alternative drug based on the entity classification.

An entity classification can be used to identify potential tests (e.g., blood panels, biopsies, gene sequencing, medical imaging, etc.) that can be performed on a patient (e.g., entity). Results from the potential tests can be recorded in a EHR (e.g., test entity record). The test results can be added as a field in the EHR.

Process 800 may include additional implementations, such as any single implementation or any combination of implementations described below and/or in connection with one or more other processes described elsewhere herein.

Although FIG. 8 shows example blocks of process 800, in some implementations, process 800 may include additional blocks, fewer blocks, different blocks, or differently arranged blocks than those depicted in FIG. 8. Additionally, or alternatively, two or more of the blocks of process 800 may be performed in parallel.

### V. Example Applications

Example applications are included to show how SPOKE can be used to classify biological entities. Section A “classification without SPOKEsigs” includes examples where SPOKE can be used to classify entities without personalized patient vectors (e.g., SPOKEsigs). Section B “classification with SPOKEsigs” is an example of how SPOKEsigs can be used to train a model to classify data from individual patients (e.g., entities).

A. Classification without SPOKEsigs

Classification can be performed using SEPs and PSEVs without the need for personalized patient vectors.

### 1. Prioritization of Drugs for Repurposing

The cost of developing a new therapeutic drug has been estimated at 1.4 billion dollars, the process typically takes 15 years from lead compound to market, and the likelihood of success is stunningly low. Strikingly, the costs have been doubling every 9 years since 1970, a sort of inverse Moore's law, which is far from an optimal strategy from both a business and public health perspective. Drug repurposing-identifying novel uses for existing therapeutics—can drastically reduce the duration, failure rates, and costs of approval. These benefits stem from the rich preexisting information on approved drugs, including extensive toxicology profiling performed during development, preclinical models, clinical trials, and postmarketing surveillance.

Modern computational approaches offer a convenient platform to tie these developments together as the reduced cost and increased velocity of in silico experimentation massively lowers the barriers to entry and price of failure.

An algorithm originally developed for social network analysis was applied it to SPOKE (Hetionet v1.0) to identify patterns of efficacy and predict new uses for drugs via edge prediction. Our approach represents an in silico implementation of network pharmacology that natively incorporates polypharmacology and high-throughput phenotypic screening. On SPOKE, the algorithm can learn which types of compound-disease paths discriminate treatments from non-treatments in order to predict the probability that a compound treats a disease.

A drug classification process can include generating DWPC metric features for compound-disease node pairs by measuring the prevalence of a metapath between a given source node and a target node. The features can be calculated by extracting paths, along the metapath, from the source node to the target node. The extracted paths can be weighted by taking the product of the node degrees along the path raised to a negative exponent (e.g., dampening exponent). The node degree, or connectivity, for a node can be the number of edges connected to a given node. The dampening exponent can determine the extent that paths through high-degree nodes are downweighted. For example the dampening exponent can be 0.4. The nodes that are annotated with the DWPC metric can be used to train a machine learning model to classify compound-disease pairs.

Accordingly, the entity can be a drug, with the drug record specifying properties of the drug. The output labels can be a probability that a given drug treats a process (e.g., demyelination) or a disease (e.g., multiple sclerosis) The SEPs can then be determined from the fields of a training set of drug records; these SEPs would likely be different than SEPs for a different training set (e.g., for patient records) that are used to classify a different type of entity. Once the SEPs are identified, the PSEVs for this training set can then be determined. The machine learning model can then be trained on the signature vectors and the known classifications for the training set of drug records.

Once a machine learning model has been trained to classify a drug based on whether it treats a particular disease, new drugs, with an unknown treatment classification, can be categorized by the model. Through this categorization process, existing drugs can be repurposed to treat new diseases.

### 2. Integrating Biomedical Research and Electronic Health Records to Create Knowledge-Based Biologically Meaningful Machine-Readable Embeddings

We described a method for embedding clinical features from more than 800,000 individuals EHRs at UCSF onto SPOKE. By connecting EHRs to SPOKE we are providing real-world context to the network thus enabling the creation of biologically and medically meaningful “barcodes” (i.e. embeddings, PSEVs) for each medical variable that maps onto SPOKE. We show that these barcodes can be used to recover purposely hidden network relationships such as Disease-Gene, Disease-Disease, Compound-Gene, and Compound-Compound. Furthermore, the correct inference of intentionally deleted edges connecting SideEffect to Anatomy nodes in SPOKE is also demonstrated.

We embed EHRs onto the SPOKE knowledge network utilizing a modified version of PageRank, the well-established random walk algorithm. These embeddings, called Propagated SPOKE Entry Vectors (PSEVs), can be created for any group of subjects with a particular characteristic (i.e. patient cohort). We created PSEVs for patient cohorts selected using either discrete or continuous EHR variables. PSEVs are vectors in which each element corresponds to a node in SPOKE. Therefore, the length of each PSEV is equal to the number of nodes in SPOKE. Furthermore, the value of each element in a PSEV encodes the importance of its corresponding node in SPOKE for a given patient cohort.

The potential uses of PSEVs are vast. We recognize that several associations in EHRs can be uncovered using clinical features alone, and several machine-learning approaches are already being utilized to that end. However, since PSEVs describe clinical features on a deeper biological level, they can be used to explain why the association is occurring in terms of Genes, Pathways, or any other nodes in a large knowledge network like SPOKE. Consequently, PSEVs can be paired with machine learning to discover new disease biomarkers, characterize patients, and drug repurposing. With implementation of some of these features, we anticipate that PSEVs or similar methods will constitute a critical tool in advancing precision medicine.

As described above, PSEVs (propagated entry vectors) are generated after identifying SEPs (entity-specific entry nodes) from EHRs (entity records) and SPOKE (graph database). PSEVs encode each node's importance to a given cohort of patients and PSEVs are aggregated to generate SPOKEsigs.

3. Knowledge Network Embedding of Transcriptomic Data from Spaceflown Mice Uncover Signs and Symptoms Associated with Terrestrial Diseases

We integrated data from six different NASA GeneLab (genelab.nasa.gov) datasets in SPOKE to enable normalization that highlighted new nodes defining systems and effects that are known to be relevant for space travel, but would have been impossible to uncover without using SPOKE. These results suggest that SPOKE can be utilized to gain a deeper biological understanding of the health hazards associated with spaceflight and provide the proof of concept for its broader utilization to integrate space and terrestrial biological data.

The samples were taken from three distinct anatomical sites (thymus, liver, and spleen) and covered multiple spaceflight durations and gravity conditions. Statistical analysis using only gene expression data illustrated that most of the differences between the samples could be attributed to either the study or the anatomical site.

Next, we hypothesized that, though this data came from a diverse set of experiments, SPOKE embeddings (i.e. “signatures” or PESVs) could be used to recover space travel changes that are conserved across the studies. SEPs were generated for mouse genes that were analogous to human genes represented by nodes in SPOKE. Mouse gene expression data (e.g., log₂ fold change (FC) data) was mapped to human gene nodes in SPOKE. If multiple mouse genes mapped to a human gene, then the average FC was used. For studies that contained multiple comparisons between spaceflown mice and control mice. In these instances, genes were removed if the FC comparisons were not in the same direction (e.g., if space versus ground day 29 had a positive FC and days 53-56 had a negative FC).

PSEVs from all of the studies were then pooled together and separated into three groups based on the type of fold change comparison (Ground vs. Baseline, Space vs. Baseline, and Space vs. Ground). Top nodes were enriched for nodes for phenotypes and physiological changes known to be impacted by spaceflight. By using gene expression as an input to a machine learning model, other physical changes resulting from spaceflight, such as symptoms and side effects, can be identified. Furthermore, paths were found between the input gene set and the top node set. These paths shed light onto the underpinnings of spaceflight related health hazards and could potentially be used to identify drug targets. In the future, archived spaceflight and other experimental samples could be used to validate the predicted signatures and assess their physiological significance without the need for further experiments. Thus, we anticipate that our results are the very first steps towards a broader collaboration utilizing the SPOKE model to compare spaceflight and terrestrial phenotypes.

In this case, the entities are various tissue samples taken from spaceflown mice. PSEVs were generated for each entity, pooled into groups and summed based on the entity's known classification (time in space). Analyzing the three groups of PSEVs with known classifications allowed for new classifications, such as phenotypic changes associated with spaceflight, to be identified. The identification of new classifications, such as phenotypic changes associated with spaceflight, illustrates how the SPOKE graph database can be used to train a model to classify a biological entity. The creation of PSEVs (propagated entry vectors) for biological entities, in this case tissue samples, ensures that genes relevant to spaceflight are identifiable by a trained model.

B. Classification with SPOKEsigs

While SPOKE, without SPOKEsigs, can be used to train models to make general predictions across a patient population, SPOKEsigs can be used to make specific predictions about a patient based on the patient's EHRs. For instance, an algorithm trained on SPOKE using SEPs and PSEVs can classify whether a drug is a potential treatment for a disease. An algorithm trained using patient specific profile vectors (e.g., SPOKEsigs) can classify whether a drug is a potential treatment for a disease as presented in a specific patient.

In this case, SPOKEsigs were used to train a model to identify patients before they are diagnosed with multiple sclerosis (MS). MS is a chronic, autoimmune disease of the central nervous system (CNS) with severe and life-long consequences. Early symptoms of MS, such as fatigue or depression, are often non-specific, which can make it difficult for the general practitioner to identify and refer the patient to a neurologist. Previous studies suggest that health care utilization increases even 10 years prior to MS diagnosis. Since early treatment of MS is associated with improved long-term neurological outcomes, early recognition of a (sub)clinical presentation and understanding the biological underpinnings of MS could have a major impact disease trajectories. This embodiment can improve early diagnosis of MS by using a new method that combines traditional statistical EHR data analytics with structured biological knowledge from a graph database.

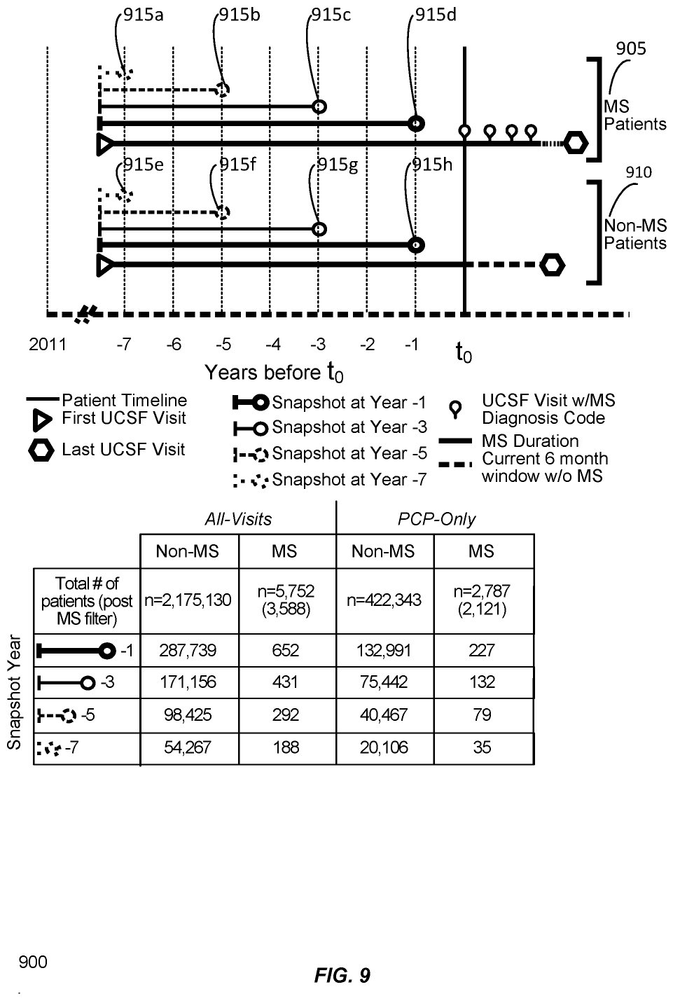

FIG. 9 shows a timeline 900 comparing a cohort of MS patients and a cohort of non-MS patients. EHR data was collected for two patient cohorts: patients diagnosed with untreated MS 905 and patients without a MS diagnosis 910. The cohorts were organized into snapshots 915a-h based on the length of available EHR data for each patient with snapshots for one year 915d 915h, three years 915c 915g, five years 915b 915f, and seven years 915a 915e of data. For instance, MS patients with at least one year of medical history were included in snapshot 915d and non-MS patients with at least three years of EHR data were included in snapshot 915g. Medical records were transformed into Observational Medical Outcomes Partnership (OMOP) common data model (CDM) so that consistent codes and terminology were used throughout the records. SEPs were found, and PSEVs were generated for each SEP, and PSEVs were generated for each patient using the techniques disclosed in section III.

Creating training vectors for subclinical MS patients is an example of how SPOKEsigs (training vectors) can be used to categorize biological entities. The MS patients have a known classification (the eventual MS diagnosis). For patients diagnosed with MS, SPOKEsigs can be generated using EHRs from time period(s) prior to their diagnosis, and a machine learning model can be trained to diagnose a disease earlier than with conventional diagnostic methods (classify a biological entity). Once the machine learning model has been trained, the model can be used to screen for MS by creating entity-specific vectors and inputting the vectors into the trained model.

### VI. Machine Learning

The potential uses of PSEVs are vast. Several associations in EHRs can be uncovered using clinical features alone, and several machine-learning approaches are already being utilized to that end. However, since PSEVs describe clinical features on a deeper biological level, they can be used to explain why the association is occurring in terms of Genes, Pathways, or any other nodes in a large knowledge network like SPOKE. Consequently, PSEVs can be paired with machine learning to discover new disease biomarkers, characterize patients, and drug repurposing. With implementation of some of these features, it is likely that PSEVs or similar methods will constitute a critical tool in advancing precision medicine.

### A. Training a Machine Learning Model

Once the SPOKEsigs are created, these vectors can be used to train a model to predict a desired outcome (e.g., selection from one or more known classifications for a new sample). A training sample includes a set of SPOKEsig and a known classification (e.g., trait) for the EHR. The particular EHR for a given patient can be at a particular instance in time, where the patient has a known classification at that time. The classification can change over time. Thus, multiple training samples can correspond to the same patient, with the EHR and classification for the training sample being at a particular snapshot in time.

The training samples are used to train the model. Accordingly, the SPOKEsigs can be used in conjunction with machine learning techniques (e.g. random forests, support vector machine, artificial neural networks, etc) to optimize a cost/loss function, to get the parameters of the classification model. As part of the training, the classification model is tuned, by updating model variables, until the predictive model accurately categorizes the training set.

An entire set of SPOKEsigs can be split into a training set and a test set. The test set is not used during the training and will be used later to evaluate if the model, developed only with the training set, makes accurate predictions. If the model accurately categorizes the evaluation set, the model can be extended to categorize new data sets.

### B. Using the Trained Model

Once the model is trained, a new entity record (e.g., an EHR) can be classified, (e.g., whether a patient has a particular disease or whether a drug is suitable for a target).

Using the new entity record, each SEP corresponding to the entity record is identified. Then, a PSEV is determined for the SEPs, and the entity signature is obtained by aggregating the corresponding PSEVs. The entity signature is then used an input feature vector for the classification model.

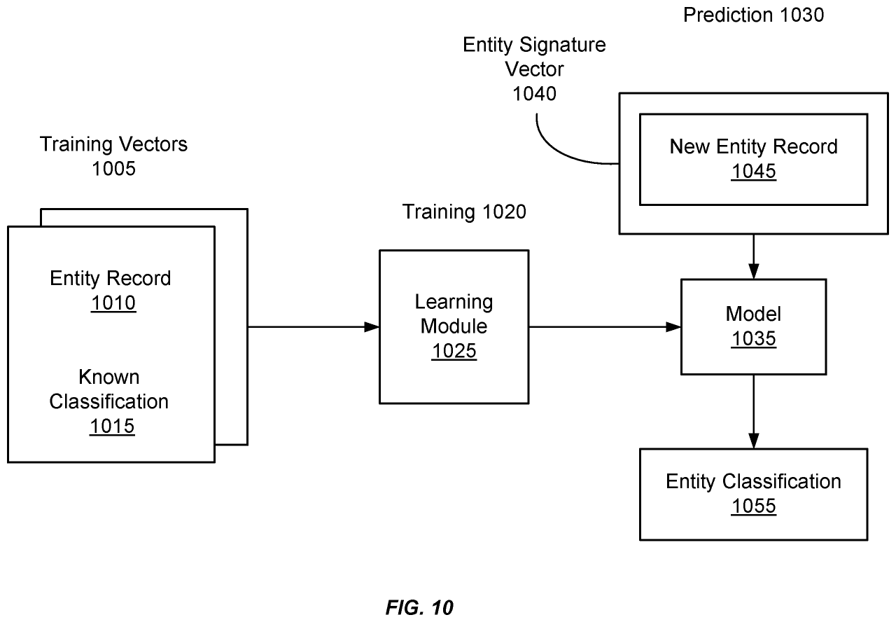

FIG. 10 depicts a machine learning model according to the embodiments of the present invention. Training vectors (SPOKEsigs) 1005 are shown with entity record 1010 and a known classification 1015. As examples, an entity could be a patient, a drug, a tissue sample, an organism, an organ, or a disease. Entity record 1010 can include various fields. For ease of illustration, only two training samples are shown, but the number of training samples may be much larger, e.g., 10, 50, 100, 1,000, 10,000, 100,000, or more. Training vectors could be made for different entities, the same entity over different time periods or at different points in disease progression (e.g. before clinical presentation).

Entity records 1010 have entity fields that can correspond to standardized medical terminology and the skilled person will appreciate the various ways that data is recorded in an electronic health record. For example entity record fields could include lab results, diagnosis table, and medication orders. Lab results could include the lab orders, the actual measurements, and a judgment of whether the results were normal. Known classifications 1015 include any entity record field. For instance, a cohort could be created for entities with a particular diagnosis (e.g. epilepsy), symptom (e.g. heartburn), or medication order (e.g. Aspirin). Example classifications can include categorization of an entity, e.g., discrete classification of whether an entity has cancer or not or continuous categorization providing a probability (e.g., a risk or a score) of a discrete value. The classification can have arbitrary support (e.g., a real number) or be an element of a small finite set. The classification can be ordinal, and thus the support can be provided as an integer. Accordingly, a classification can be categorical, ordinal, or real, and can relate to a single measurement or multiple measurements, and may be high dimensional.

Training vectors 1005 can be used by a learning module 1025 to perform training 1020. Learning module 1025 can optimize parameters of a model 1035 such that a quality metric (e.g., accuracy of model 1035) is achieved with one or more specified criteria. The accuracy may be measured by comparing known classifications 1015 to predicted classifications. Parameters of model 1035 can be iteratively varied to increase accuracy. Determining a quality metric can be implemented for any arbitrary function including the set of all risk, loss, utility, and decision functions.

In some embodiments of training, a gradient may be determined for how varying the parameters affects a cost function, which can provide a measure of how accurate the current state of the machine learning model is. The gradient can be used in conjunction with a learning step (e.g., a measure of how much the parameters of the model should be updated for a given time step of the optimization process). The parameters (which can include weights, matrix transformations, and probability distributions) can thus be optimized to provide an optimal value of the cost function, which can be measured as being above or below a threshold (i.e., exceeds a threshold) or that the cost function does not change significantly for several time steps, as examples. In other embodiments, training can be implemented with methods that do not require a hessian or gradient calculation, such as dynamic programming or evolutionary algorithms.

A prediction stage 1030 can provide a predicted entity classification 1055 for a new entity's entity signature vector 1040 based on new entity records 1045. The new entity records can be of a similar type as entity record 1010. If new entity records are of a different type, a transformation can be performed on the data to obtain data in a similar format as entity record 1010. Ideally, predicted entity classification 1055 corresponds to the true entity classification for new entity signature vector 1040.

Examples of machine learning models include deep learning models, neural networks (e.g., deep learning neural networks), kernel-based regressions, adaptive basis regression or classification, Bayesian methods, ensemble methods, logistic regression and extensions, Gaussian processes, support vector machines (SVMs), a probabilistic model, and a probabilistic graphical model. Embodiments using neural networks can employ using wide and tensorized deep architectures, convolutional layers, dropout, various neural activations, and regularization steps.

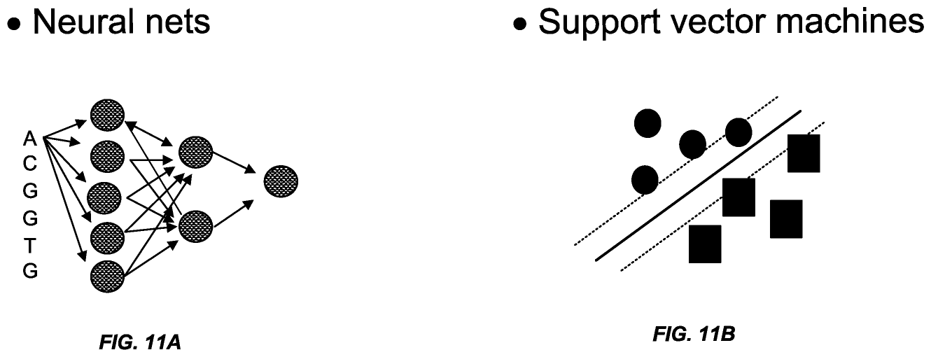

FIG. 11A shows an example machine learning model of a neural network. As an example, model 1135 can be a neural network that comprises a number of neurons (e.g., Adaptive basis functions) organized in layers. The training of the neural network can iteratively search for the best configuration of the parameter of the neural network for feature recognition and classification performance. Various numbers of layers and nodes may be used. A person with skills in the art can easily recognize variations in a neural network design and design of other machine learning models.

FIG. 11B shows an example machine learning model of a support vector machine (SVM). As another example, model 1135 can be a support vector machine. Features can be treated as coordinates in a coordinate space. Samples of training data points (e.g., multidimensional data points composed of the measured data). The training data points are distributed in the space, and the support vector machine can identify boundaries between the classifications.

### VII. Computer System

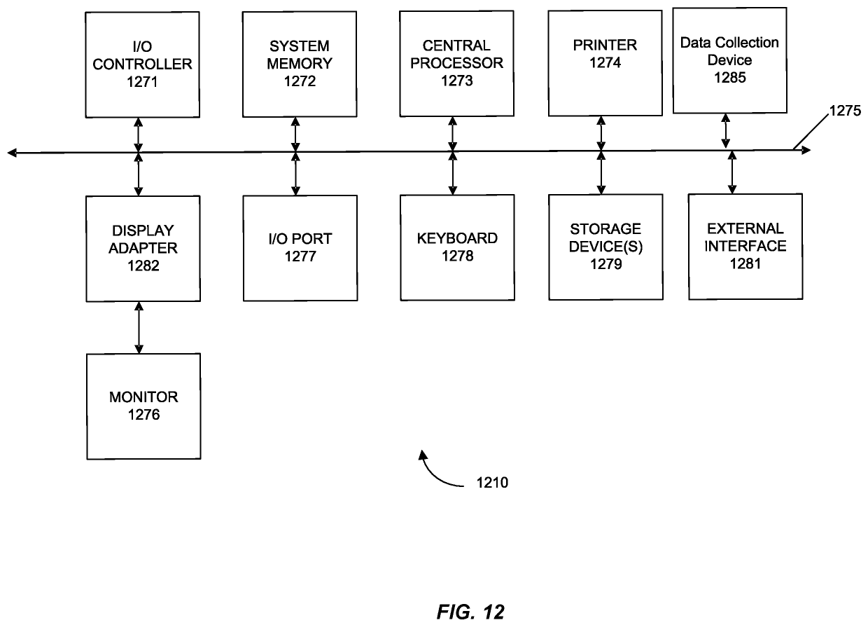

Any of the computer systems mentioned herein may utilize any suitable number of subsystems. Examples of such subsystems are shown in FIG. 12 in computer system 1210. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems shown in FIG. 12 are interconnected via a system bus 1275. Additional subsystems such as a printer 1274, keyboard 1278, storage device(s) 1279, monitor 1276 (e.g., a display screen, such as a light emitting diode (LED) display screen), which is coupled to display adapter 1282, and others are shown. Peripherals and input/output (I/O) devices, which couple to I/O controller 1271, can be connected to the computer system by any number of means known in the art such as input/output (I/O) port 1277 (e.g., USB, FireWire®). For example, I/O port 1277 or external interface 1281 (e.g. Ethernet, Wi-Fi, etc.) can be used to connect computer system 1210 to a wide area network such as the Internet, a mouse input device, or a scanner. The interconnection via system bus 1275 allows the central processor 1273 to communicate with each subsystem and to control the execution of a plurality of instructions from system memory 1272 or the storage device(s) 1279 (e.g., a fixed disk, such as a hard drive, or optical disk), as well as the exchange of information between subsystems. The system memory 1272 and/or the storage device(s) 1279 may embody a computer readable medium. Another subsystem is a data collection device 1285, such as a camera, microphone, accelerometer, and the like. Any of the data mentioned herein can be output from one component to another component and can be output to the user.

A computer system can include a plurality of the same components or subsystems, e.g., connected together by external interface 1281, by an internal interface, or via removable storage devices that can be connected and removed from one component to another component. In some embodiments, computer systems, subsystem, or apparatuses can communicate over a network. In such instances, one computer can be considered a client and another computer a server, where each can be part of a same computer system. A client and a server can each include multiple systems, subsystems, or components.

Aspects of embodiments can be implemented in the form of control logic using hardware circuitry (e.g. an application specific integrated circuit or field programmable gate array) and/or using computer software stored in a memory with a generally programmable processor in a modular or integrated manner, and thus a processor can include memory storing software instructions that configure hardware circuitry, as well as an FPGA with configuration instructions or an ASIC. As used herein, a processor can include a single-core processor, multi-core processor on a same integrated chip, or multiple processing units on a single circuit board or networked, as well as dedicated hardware. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C, C++, C#, Objective-C, Swift, or scripting language such as Perl or Python using, for example, conventional or object-oriented techniques. The software code may be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk) or Blu-ray disk, flash memory, and the like. The computer readable medium may be any combination of such devices. In addition, the order of operations may be re-arranged. A process can be terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

Such programs may also be encoded and transmitted using carrier signals adapted for transmission via wired, optical, and/or wireless networks conforming to a variety of protocols, including the Internet. As such, a computer readable medium may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing a respective step or a respective group of steps. Although presented as numbered steps, steps of methods herein can be performed at a same time or at different times or in a different order. Additionally, portions of these steps may be used with portions of other steps from other methods. Also, all or portions of a step may be optional. Additionally, any of the steps of any of the methods can be performed with modules, units, circuits, or other means of a system for performing these steps.

The specific details of particular embodiments may be combined in any suitable manner without departing from the spirit and scope of embodiments of the disclosure. However, other embodiments of the disclosure may be directed to specific embodiments relating to each individual aspect, or specific combinations of these individual aspects.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

All patents, patent applications, publications, and descriptions mentioned herein are incorporated by reference in their entirety for all purposes. None is admitted to be prior art. Where a conflict exists between the instant application and a reference provided herein, the instant application shall dominate.

1. A method for generating a machine learning model using a graph database, the method comprising:
storing a graph database comprising (1) M nodes of a plurality of node types and (2) a plurality of edges of a plurality of edge types;
receiving, for a plurality of entities, a plurality of entity records, each with a plurality of fields and a known classification;
identifying N entry nodes of the M nodes that each match one of the plurality of fields, wherein N is less than M;
for each entry node of the N entry nodes, generating a propagated entry vector having M entry values, wherein each of the M entry values represents an importance of a corresponding node to the entry node;
for each of the plurality of entities:
identifying each of the fields of a set of corresponding entity records that matches one of the N entry nodes, thereby identifying K entity-specific entry nodes, wherein K is less than or equal to N;
identifying a set of K entity-specific entry vectors corresponding to the K entity-specific entry nodes;
generating a training vector by aggregating each of the K entity-specific entry vectors; and
training the machine learning model using the training vectors and the known classifications.

2. The method of claim 1, further comprising identifying a set of the corresponding entity records that matches one of the N entry nodes by:
for each entity record in the set of corresponding entity records:
determining a corresponding entity record, of the set of corresponding entity records, and an entry node, of the N entry nodes, that are linked together in a terminology database.

3. The method of claim 1, further comprising identifying K entity-specific entry nodes by:
for each of the plurality of entities:
determining a subset of the set of corresponding entity records, with a subset of fields, that were generated during a specified time period; and
identifying each of the subset of fields of the set of corresponding entity records that matches one of the N entry nodes, thereby identifying K entity-specific entry nodes, wherein K is less than or equal to N.

4. The method of claim 1, further comprising:
for each of the plurality of entities:
determining that a number of identified K entity-specific entry nodes is above an entry node threshold; and
generating a training vector by aggregating the K entity-specific entry vectors in response to the determination.

5. The method of claim 1, further comprising:
determining an objective node of the N entry nodes, the objective node corresponding to the classification;
for each of the plurality of entities:
identifying a number of the fields of the set of corresponding entity records that matches the objective node;
determining if the identified number of fields that match is above an objective threshold; and
generating a training vector by aggregating each of the K entity-specific entry vectors in response to the determination.

6. The method of claim 1, further comprising:
for each of the plurality of entities:
identifying a record source for each of the set of corresponding entity records;
identifying a subset of records, of the set of corresponding entity records, based on the record source; and
identifying each of the fields of the subset of records that matches one of the N entry nodes, thereby identifying K entity-specific entry nodes, wherein K is less than or equal to N.

7. The method of claim 1, further comprising:
for each of the plurality of entities:
determining a total number of fields of the set of corresponding entity records;
determining that the total number of fields is above a field threshold; and
generating a training vector by aggregating each of the K entity-specific entry vectors in response to the determination.

8. The method of claim 1, wherein the K entity-specific entity vectors are aggregated by summing each of the entity-specific entry vectors of the K entity-specific entry vectors.

9. A method for categorizing entity records using a machine learning model, the method comprising:
storing database data comprising N entry nodes to a graph database that includes M nodes, wherein M is greater than N;
receiving an entity record including a plurality of fields;
identifying a set of the plurality of fields of the entity record that each matches one of the N entry nodes, thereby identifying K entity-specific entry nodes, wherein K is less than or equal to N;
identifying, from a plurality of entry vectors, K entity-specific entry vectors corresponding to the K entity-specific entry nodes, wherein each of the plurality of entry vectors includes M entry values, and wherein each of the M entry values for an entry vector represents an importance of a corresponding node to an entry node for the entry vector;
generating an entity signature vector by aggregating the K entity-specific entry vectors;
inputting the entity signature vector into the machine learning model; and
receiving an entity classification for the entity record as an output from the machine learning model.

10. The method of claim 9, wherein the classifications performed by the machine learning model include classifying a compound, tissue, gene, phenotype, genotype, or disease as having an effect on one or more compounds, tissues, genes, phenotypes, genotypes, or diseases.

11. The method of claim 9, wherein identifying the N entry nodes that match one of the plurality of fields comprises:
for each of the plurality of fields:
determining an entry node, of the N entry nodes, that matches a field of the plurality of fields.

12. The method of claim 9, further comprising adding a new database to the graph database by:
accessing a graph generated from a database comprising a plurality of database nodes of a plurality of database node types and a plurality of database edges of a plurality of database edge types;
identifying a first subset of database nodes and a second subset of the M nodes that are linked together in a terminology database; and
merging the first subset of database nodes and the second subset of the M nodes.

13. The method of claim 9, further comprising identifying the set of K entity-specific entry vectors corresponding to the K entity-specific entry nodes by:
for each entity-specific entry vector:
identifying an entity-specific entry vector, of the set of K entity-specific entry vectors, that was generated using an entity-specific entry node of the K entity-specific entry nodes.

14. The method of claim 9, further comprising:
providing a treatment to a subject associated with the entity record based on the entity classification.

15. The method of claim 9, further comprising:
updating a treatment plan to a subject associated with the entity record based on the entity classification.

16. The method of claim 9, further comprising:
performing tests on a subject associated with the entity record based on the entity classification.

17. The method of claim 16, performing tests on an entity by:
generating a test entity record to record the test results.

18. A computer product comprising a non-transitory computer readable medium storing a plurality of instructions that when executed control a computer system to perform operations comprising:
storing a graph database comprising (1) M nodes of a plurality of node types and (2) a plurality of edges of a plurality of edge types;
receiving, for a plurality of entities, a plurality of entity records, each with a plurality of fields and a known classification;
identifying N entry nodes of the M nodes that each match one of the plurality of fields, wherein N is less than M;
for each entry node of the N entry nodes, generating a propagated entry vector having M entry values, wherein each of the M entry values represents an importance of a corresponding node to the entry node;
for each of the plurality of entities:
identifying each of the fields of a set of corresponding entity records that matches one of the N entry nodes, thereby identifying K entity-specific entry nodes, wherein K is less than or equal to N;
identifying a set of K entity-specific entry vectors corresponding to the K entity-specific entry nodes;
generating a training vector by aggregating each of the K entity-specific entry vectors; and
training a machine learning model using the training vectors and the known classifications.

19. (canceled)

20. (canceled)

21. (canceled)

22. (canceled)

23. The computer product of claim 18, further comprising identifying a set of the corresponding entity records that matches one of the N entry nodes by:
for each entity record in the set of corresponding entity records:
determining a corresponding entity record, of the set of corresponding entity records, and an entry node, of the N entry nodes, that are linked together in a terminology database.

24. The computer product of claim 18, further comprising identifying K entity-specific entry nodes by:
for each of the plurality of entities:
determining a subset of the set of corresponding entity records, with a subset of fields, that were generated during a specified time period; and
identifying each of the subset of fields of the set of corresponding entity records that matches one of the N entry nodes, thereby identifying K entity-specific entry nodes, wherein K is less than or equal to N.
